(19) United States
(12) Patent Application Publication
(10) Pub. No.: US 2023/0005574 A1
(43) Pub. Date: Jan. 5, 2023

(54) METHODS AND SYSTEMS FOR COMPREHENSIVE SYMPTOM ANALYSIS

(71) Applicant: University of Alaska Anchorage, Anchorage, AK (US)

(72) Inventors: Zachary Patrick Sparks, Eagle River, AK (US); Jacob Lomonaco, Seattle, WA (US); Kenneth Sparks, Anchorage, AK (US)

(73) Assignee: University of Alaska Anchorage, Anchorage, AK (US)

(21) Appl. No.: 17/773,963

(22) PCT Filed: Nov. 4, 2020
(86) PCT No.: PCT/US2020/058877

§ 371 Date: May 3, 2022

Related U.S. Application Data
(60) Provisional application No. 62/930,364, filed on Nov. 4, 2019.

(51) Int. Cl. G16H 10/20

(52) U.S. Cl. CPC G16H 10/20

(57) ABSTRACT

A patient care system may collect and analyze data regarding characteristics of an individual to identify any health, medical, and/or physiological conditions associated with the individual. Trend analysis may predict an onset of a medical condition associated with the individual. Trend analysis may determine and/or identify one or more target areas (e.g., geographical areas) where individuals experience particular symptoms and/or affected by various health, medical, and/or mental issues. An alert, diagnosis, and/or treatment plan may be determined for an individual based on a corpus of information collected from and/or associated with multiple individuals.

### CROSS-REFERENCE TO RELATED PATENT APPLICATION

This application claims priority to U.S. Provisional Application No. 62/930,364, filed on Nov. 4, 2019, incorporated by reference in its entirety herein.

### BACKGROUND

A person may experience a variety of symptoms and/or physiological issues that may directly affect their health and/or wellbeing. Symptoms and/or physiological issues that directly affect a person's health and/or wellbeing may be associated with and/or exasperated by factors associated with the person and/or the person's lifestyle, such as a person's health and/or medical history (e.g., preconditions, congenital issues, previous treatments/diagnoses, etc.), a person's occupation (e.g., military service history, type of work performed, etc.), areas and/or regions that the person may have been exposed to, and/or the like. Symptoms and/or physiological issues that directly affect someone's health and/or wellbeing may go undetected/unidentified due to a lack of awareness of the individual of such symptoms and/or physiological issues, and/or a failure of the individual to inform a clinical professional of such symptoms and/or physiological issues. Screening can improve detection of symptoms and/or physiological issues that directly affect the health and/or wellbeing of an individual. Individuals may be interviewed with standardized screening questions and their responses may be manually and/or verbally entered to a computerized patient record system. Manual and/or verbal entrance of user (e.g., patient, subject, etc.) records into medical and/or health record systems is laborious, subject to transcription and/or translation error, and/or subject to incomplete/incorrect information due to self-reporting anxiety. Further, trends and or commonalities between individuals affected by symptoms and/or physiological issues may go undetected.

### SUMMARY

It is to be understood that both the following general description and the following detailed description are exemplary and explanatory only and are not restrictive. Methods and systems for comprehensive symptom analysis are described. A user device (e.g., a mobile device, a smart device, computing device, etc.) may periodically collect and/or actively solicit a user (e.g., patient, subject, etc.) for data/information. The data/information may be associated/integrated with a health and/or medical record system and used to determine, track, and/or the like various health and medical related issues that may affect a user and/or multiple users of a similar demographic.

The user device may generate and/or display, via an interface (e.g., a graphical user interface (GUI), etc.), screening questions. Screening questions may target and/or be directed towards any users or users of a particular demographic, such as the general veteran population. The screening questions may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. For example, screening questions may be associated with the military service history of a user, somatic symptoms a user may be experiencing, pain/injury a user may be experiencing, illness a user may be experiencing, personal and/or social behavior of the user, depression symptoms the user may be experiencing, and/or the like.

A user profile may be generated/created based on responses to the screening questions. A user profile may include a baseline metric and/or dataset based on the responses to the screening questions. In some cases, additional data/information that track symptoms, activity, diet, and/or vitals of a user may be collected from one or more devices associated with the user device and/or the user, such as an accelerometer, a pedometer, a geographical position sensing (GPC) module, an oximeter, a tactile sensor, and/or the like. For example, data/information (e.g., one or more signals, etc.) from an accelerometer, a pedometer, a geographical position sensing (GPC) module, and/or a tactile sensor may be analyzed (e.g., a long-term evaluation, etc.) to determine movement and impact velocities associated with the user device and/or the user. The data/information may be associated with the user profile (e.g., baseline metric and/or dataset, responses to screening questions, etc.). In some cases, the user profile may include additional data/information from one or more records relative to the user, such as an electronic medical record, a military service record, and/or the like. The user profile may be used to determine/predict the onset of and/or a change to any health, medical, and/or mental issues the user may be experiencing based on one or more symptomatic indicators. A symptomatic indicator may indicate a likelihood that the user (e.g., patient, subject, etc.) may be experiencing and/or be affected by a health issue, a medical issue, a physiological issue, a psychological issue, and/or the like.

In some instances, a plurality of user profiles associated with a plurality of users may be used to determine and/or identify one or more target areas (e.g., geographical areas) where individuals likely may be affected by a health, medical, or mental issue associated with one or more symptomatic indicators. Comprehensive symptom analysis may improve health care delivery and reduce cost related to health care by tracking symptoms, activity, diet, and/or vitals of a user to by enabling clinicians to evaluate the health, medical, and/or mental state of a user.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, together with the description, serve to explain the principles of the methods and systems:

FIG. 1 shows an example system;

FIG. 2 shows an example machine learning system;

FIG. 3 shows an example graphical user interface (GUI);

FIG. 4 shows a flowchart of an example method;

FIG. 5 shows a flowchart of an example method; and

FIG. 6 shows a block diagram of an example computing device.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

Methods and systems for comprehensive symptom analysis are described. A user device (e.g., a mobile device, a smart device, computing device, etc.) may periodically collect and/or actively solicit a user (e.g., patient, subject, etc.) for data/information. The data/information may be associated and/or integrated with a health and/or medical record of a user to determine, track, and/or the like various health and/or medical related issues that may affect a user and/or multiple users of a similar demographic.

The user device may generate and/or display, via an interface (e.g., a graphical user interface (GUI), etc.), screening questions. Screening questions may target any user or may be directed towards users of a particular demographic, such as the general veteran population. Screening questions may target and/or be directed towards any user. Screening questions may be associated with and/or include one or more symptomatic indicators. A symptomatic indicator may indicate a likelihood that the user (e.g., patient, subject, etc.) may be experiencing and/or be affected by a health issue, a medical issue, a physiological issue, a psychological issue, and/or the like. Screening questions may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. For example, screening questions may be associated a military service history of a user, somatic symptoms a user may be experiencing, pain/injury a user may be experiencing, illness/irritability a user may be experiencing, personal and/or social behavior associated with a user, depression/anxiety/paranoia symptoms a user may be experiencing, and/or the like. Screening questions may be associated with any topic, health/medical related issue, and/or the like relative to a user.

For each response (e.g., tactile response, audio response, patient provided response, etc.) to the screening questions and/or symptomatic indicators, a score (e.g., a score on a scale from 1-10, etc.) may be determined. In some instances, the user device may determine scores to the screening questions, such as by accessing data/information associated with the screening questions and/or symptomatic indicators and correlating each response to the screening questions and/or symptomatic indicators to the data/information associated with the screening questions and/or symptomatic indicators. In some instances, the user device communicate with a computing device (e.g., cloud-based device, server, electronic records management device, etc.) and provide the responses to the screening questions to the computing device. The computing device may determine scores to the screening questions. Scores to the screening questions may be determined, for example, by accessing data/information associated with the screening questions (and/or symptomatic indicators) and correlating each response to the screening questions (and/or symptomatic indicators) to the data/information associated with the screening questions (and/or symptomatic indicators). For example, a machine learning module may be used to correlate each response to the screening questions (and/or symptomatic indicators) to data/information associated with the screening questions (and/or symptomatic indicators).

Data/information associated with the screening questions (and/or symptomatic indicators) may be and/or associated with a scale/score. For example data/information associated with physical injury, illness, and pain (PIIP) may be associated with a scale. For example, a screening question may ask the user where they are experiencing pain and to describe the type of pain (e.g., aching, burning, cramping, dull, earache, fatigue, gastrointestinal pain, headache, itching, sharp, stabbing, shocking, etc.). The screening questions may be derived from a catalogue and/or data repository of known diseases, illnesses, and injuries to enable the user to describe in great diagnostic detail what they are experiencing. The user may provide a response based on a scale from 1-10, where scores/values of 4 or greater are considered to be clinically significant pain, and/or the like. The data/information associated with the screening questions and/or symptomatic indicators may be and/or associated with any scale and/or score. Each response to the screening questions and/or symptomatic indicators may be associated with a score that is determined relative to a scale and/or the like associated with each screening questions. Each score generated and/or determined based on responses to the screening questions may be associated with user information (e.g., a user profile, an electronic medical record (EMR) associated with the patient, a user account, etc.).

Data/information associated with the screening questions may include data indicative of keystrokes and/or answer selections made by a user. For example, a user may initially select a first answer to a screening question (e.g., a selection of “4 out of 10” on a pain scale), and the user may subsequently select (e.g., 3 second after selecting the first answer) a second answer question to the screening question (e.g., a selection of “6 out of 10” on a pain scale). The data indicative of the keystrokes and/or the answer selections made by the user may be used to adjust for patient self-reporting anxiety. For example, the data/information associated with physical injury, illness, and pain (PIIP) may be adjusted based on the data indicative of the keystrokes and/or the answer selections.

User information (e.g., a user profile, an electronic medical record (EMR) associated with the patient, a user account, etc.) may include a baseline metric and/or dataset based on the responses to the screening questions, previous responses to screening questions. The baseline metric and/or dataset may be adjusted, modified, and/or changed by any collected data/information regarding the activity, diet, and/or vitals of the user. In some cases, additional data/information may be collected from one or more devices associated with the user device and/or user, such as an accelerometer, a pedometer, a geographical position sensing (GPC) module, an oximeter, a tactile sensor, and/or the like. For example, data/information (e.g., one or more signals, etc.) from an accelerometer, a pedometer, a geographical position sensing (GPC) module, and/or a tactile sensor may be analyzed (e.g., a long-term evaluation, etc.) to determine movement and impact velocities associated with the user device and/or the user. The data/information (e.g., one or more signals, etc.) may be associated with the user information (e.g., baseline metric and/or dataset, responses to screening questions, etc.). In some cases, the user information may include data/information from one or more records systems such as an electronic medical record, a military service record, and/or any other record/information associated with the user.

In some instances, the user device may solicit data/information associated with the user. For example, the user profile may be analyzed to determine a time (e.g., instance, date, period, etc.) when responses to screening questions and/or data/information from the one or more devices (e.g., an accelerometer, a pedometer, a geographical position sensing (GPC) module, an oximeter, a tactile sensor, etc.) associated with the user device was received. The user device (and/or a computing device) may determine, based on the time when the responses to the screening questions and/or the data/information from the one or more devices was received, a time frame, a time period, a time window, and/or the like. The time frame (e.g., time period, time window, etc.) may be relative to when additional responses to screening questions and/or additional data/information from the one or more devices should be received, such as daily, weekly, monthly, and/or the like. The time frame may be automatically determined based on the user profile and/or any symptomatic indicators affecting the user. The time frame may be determined by the user. When additional responses to screening questions and/or additional data/information from the one or more devices is not received within the time frame, the user device (and/or computing device) may send a notification (e.g., a signal, a message, an email, a text, etc.) to the user device and/or the user that causes the user to provide additional responses to screening questions and/or additional data/information from the one or more devices.

The user profile may be used to determine/predict the onset of and/or a change to any health, medical, and/or mental issues the user may be experiencing based on one or more symptomatic indicators. Each score generated and/or determined based on responses to the screening questions may be used to determine/predict the onset of and/or a change to any health, medical, and/or mental issues, such as a likelihood that the user will be diagnosed with a health/medical issue. In some instances, the user device and/or computing device (e.g., via the machine learning module) may determine the likelihood that a user will be diagnosed with a health/medical issue based on a score generated and/or determined from responses to the screening questions. Scores satisfying a threshold (e.g., a clinical threshold, etc.) may indicate a health issue. For example, scores may be used to determine an alert condition.

An alert condition may be an indication that a user (e.g., patient, subject, etc.) is at risk for an emergency medical condition. An alert/notification (e.g., a clinical notification and/or reminder, etc.) may be determined and/or generated based on an alert condition. For example, a score determined from a response to a screening question associated with pain (e.g., aching, burning, cramping, dull, earache, fatigue, gastrointestinal pain, headache, itching, sharp, stabbing, shocking, etc.) may exceed a threshold value of 4 (on a scale from 1-10), and may alert condition to be determined and/or an alert/notification to be generated/sent (e.g., sent to a medical and/or healthcare device/professional, etc.).

In some instances, a plurality of user profiles associated with a plurality of users may be used to determine and/or identify one or more target areas (e.g., geographical areas) where individuals likely may be associated with one or more symptomatic indicators. For example, the plurality of user profiles may be analyzed (via a machine learning module) to determine not only target areas (e.g., geographical areas) where individuals experience a high rate of suicide, but also why a target area has such a high suicide rate for users that may have been previously stationed in the target area during military service.

The methods and systems for comprehensive symptom analysis described herein may lower the cost of medical care and reduce the analytical burdens on clinicians faced with increasing amounts of clinical data. Comprehensive symptom analysis may improve health care delivery and reduce cost related to health care by tracking symptoms, activity, diet, and/or vitals of a user to by enabling clinicians to evaluate the health, medical, and/or mental state of a user. The methods and systems for comprehensive symptom analysis described may save time, improved data capture, and improve early detection of mental issues and/or disorders. The methods and systems for comprehensive symptom analysis described may aid triage and referral of patients to healthcare and/or medical professionals, lower the cost of medical care, and reduce the analytical burdens associated with systematic screening (e.g., manual and/or verbal entrance of patient records into patient record systems, etc.).

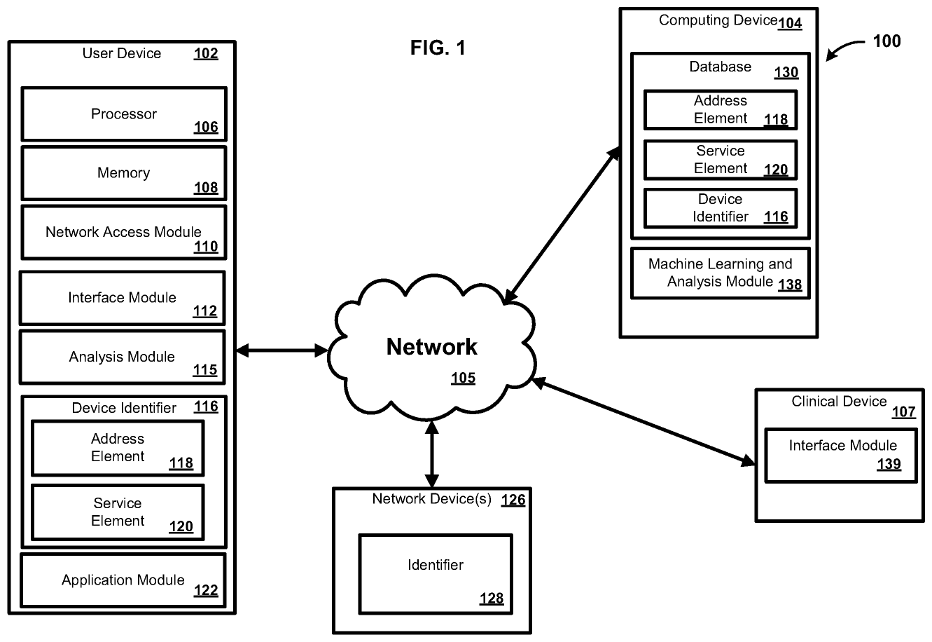

FIG. 1 shows a system 100 for comprehensive symptom analysis. One skilled in the art will appreciate that provided herein is a functional description and that the respective functions may be performed by software, hardware, or a combination of software and hardware. The system 100 may include a network 105. The network 105 may be a private and/or public network, such as the Internet, a local area network, a wide area network, a cellular network, a satellite network, combinations thereof, and/or the like. The network 105 may include and/or support any form of wired and/or wireless communication.

The system 100 may include one or more network devices 126. The one or more network device(s) 126 may facilitate the connection of a device, such as a user device 102, to the network 105. The one or more network device(s) 126 may be part of a cellular network. The one or more network device(s) 126 may be and/or include a wireless access point (WAP). The network device(s) 126 may allow one or more wireless devices to connect to a wired and/or wireless network using Wi-Fi, Bluetooth or any desired method or standard. The network device(s) 126 may be and/or include a dual band wireless access point. The network device(s) 126 may be configured with a first service set identifier (SSID) (e.g., associated with a user network or private network) to function as a local network for a particular user or users. The network device(s) 126 may be configured with a second service set identifier (SSID) (e.g., associated with a public/community network or a hidden network) to function as a secondary network or redundant network for connected communication devices.

The network device(s) 126 may include an identifier 128. One or more identifiers (e.g., the identifier 128, etc.) may be or relate to an Internet Protocol (IP) Address IPV4/IPV6 or a media access control address (MAC address) or the like. The identifier 128 may be a unique identifier for facilitating communications on a physical network. The network device(s) 126 may include a distinct identifier 128 that is associated with a physical location of the network device(s) 126.

The system 100 may include a user device 102 (e.g., a mobile device, a smart device, a computing device, etc.) in communication with a computing device 104 (e.g., cloud-based device, server, electronic records management device, etc.) and/or a clinical device 107. In some instances, the system 100 may include multiple user devices (e.g., user device 102) in communication with the computing device 104 and/or multiple clinical devices (e.g., clinical device). The user device 102 may be in communication with the computing device 104 and/or a clinical device 107 via a long-range communication technique (e.g., Internet, cellular, satellite, and the like), via a short-range communication technique (e.g., BLUETOOTH®, ZigBee, Z-wave, near-field communication, infrared, etc.), and/or via any communication technique.

The computing device 104 and/or a clinical device 107 may be disposed locally or remotely relative to the user device 102. The user device 102 and the computing device 104 can be in communication via the network 105. In some instances, the system 100 may include multiple user devices (e.g., the user device 102, etc.), computing devices (e.g., the computing device 104, etc.), and/or clinical devices (e.g., the clinical device 107, etc.) in communication via the network 105.

The user device 102 may be associated with a user identifier or device identifier 116. The device identifier 116 may be and/or include a mobile directory number (MDN), a mobile identification number (MIN), an international mobile subscriber identity (IMSI), an international mobile equipment identifier (IMEI), and/or the like. The device identifier 116 may be and/or include any identifier, token, character, string, and/or the like, for differentiating one user or user device (e.g., user device 102) from another user or user device. The device identifier 116 may identify a user or user device as belonging to a particular class of users or user devices. The device identifier 116 may comprise information relating to the user device 102 such as a manufacturer, a model or type of device, a service provider associated with the user device 102, a state of the user device 102, a locator, and/or a label or classifier. The device identifier 116 may comprise and/or be associated with information relating one or more applications installed on and/or associated with the user device 102. Other and/or any information may be represented by the device identifier 116.

The device identifier 116 may comprise an address element 118 and a service element 120. The address element 118 may include or provide a mobile directory number (MDN), an internet protocol address, a network address, a media access control (MAC) address, an Internet address, and/or the like. The address element 118 may be relied upon to establish a communication session between the user device 102, the computing device 104, the clinical device 107, and/or any other device/network/system. The address element 118 may be used as an identifier or locator of the user device 102. The address element 110 may be persistent for a particular network.

The service element 120 may comprise an identification of a service provider associated with the user device 102 and/or with the class of user device 102. The class of the user device 102 may be related to a type of device, capability of device, type of service being provided, and/or a level of service (e.g., business class, service tier, service package, etc.). The service element 120 may comprise information relating to or provided by a communication service provider (e.g., an application service provider, an Internet service provider) that is providing or enabling data/information flow such as application (e.g., a software application, etc.) and/or communication services to the user device 102. The service element 120 may comprise information relating to a preferred service provider for one or more particular services relating to the user device 102. The address element 118 may be used to identify or retrieve data/information from the service element 120, or vice versa. The address element 118 and the service element 120 may be stored remotely from the user device 102 and retrieved by one or more devices such as the user device 102 and/or the computing device 104. Other information may be represented by the service element 120.

The user device 102 can comprise a processor 106. The processor 106 may be and/or include any suitable microprocessor or microcontroller, such as a low-power application-specific controller (ASIC) and/or a field programmable gate array (FPGA) designed or programmed specifically for the task of controlling the user device 102 as described herein, or a general purpose central processing unit (CPU) (e.g., a CPU based on 80×86 architecture as designed by Intel™ or AMD™, or a system-on-a-chip as designed by ARM™. The processor 106 can be coupled to auxiliary devices or modules of the user device 102 via a bus or other coupling.

The user device 102 include a non-transitory memory module 108 coupled to the processor 106. The memory 108 can comprise a random access memory (RAM) for storing program instructions and data/information for execution and/or processing by the processor 106 during control of the user device 102. The memory module 108 may store user (e.g., patient, subject, etc.) data/information, and/or screening information (e.g., one or more symptomatic indicators relating to patient health and/or mental status). Screening information may be any data/information used to elicit information and/or responses associated with user health (e.g., mental health), status, and/or wellbeing. For example, screening information may include, but is not limited to, screening questions and/or symptomatic indicators. Screening questions may target and/or be directed towards users of a particular demographic, such as the general veteran population. Screening questions may target and/or be directed towards any user.

When the user device 102 is powered off and/or in an inactive state, screening information, program instructions, and/or any other data/information may be stored in a long-term memory, such as a non-volatile magnetic optical, an electronic memory storage device (not shown), and/or the like. The RAM and/or the long-term memory may store and/or include one or more application programming interfaces (APIs) associated with one or more applications associated with and/or installed on the user device 102, such as an application associated with comprehensive symptom analysis. The RAM and/or the long-term memory may include a non-transitory computer-readable medium storing program instructions that, when executed by the processor 106, cause the user device 102 to perform all or part of one or more methods and/or operations described herein. Program instructions and/or the like may be written in any suitable high-level language, such as C, C++, C#, Java™ and/or the like. Program instructions and/or the like may be compiled to produce machine-language code for execution by the processor 106.

The user device 102 can include a network access module 110. The network access module 110 may enable the user device 102 to be coupled to and/or in communication with one or more ancillary devices such as via a network device 126 (e.g., a access point, etc.) associated with a wireless telephone network, local area network, service provider, the Internet, and/or the like. The user device 102 (processor 106) may share data/information (e.g., comprehensive symptom analysis data/information, etc.) with the one or more ancillary devices via the network access module 110. The shared data/information can comprise application data/information, call data/information, messaging data/information, usage data/information, location data/information, operational data/information associated with the user device 102, a status of the user device 102, a status and/or operating condition of one or more the components of the user device 102, text to be used in a message, and/or any other data. The user device 102 may be configured to receive control instructions from the one or more ancillary devices via the network access module 110. A configuration of the user device 102, an operation of the user device 102, and/or any other settings of the user device 102, may be controlled by the one or more ancillary devices, such as another user device 102 and/or the computing device 104, via the network access module 110.

The user device 102 may include an interface module 112. The interface module 112 may include and/or be associated with a communication interface such as a web browser (e.g., Internet Explorer, Mozilla Firefox, Google Chrome, Safari, or the like). Other software, hardware, and/or interfaces can be used to provide communication between the user and one or more of the user device 102, the computing device 104, the clinical device 107, and/or any other device. The interface module 112 can request or query various files from a local source and/or a remote source, such as data/information associated with and/or including screening questions and/or symptomatic indicators. Screening questions may be associated with and/or include one or more symptomatic indicators. A symptomatic indicator may indicate a likelihood that the user (e.g., patient, subject, etc.) may be experiencing and/or be affected by a health issue, a medical issue, a physiological issue, a psychological issue, and/or the like.

The interface module 112 may provide an interface to a user to interact with the user device 102, the computing device 104, the clinical device 107, and/or any other device. The interface module 112 can include any interface for presenting information to a user, such as one or more visual interfaces (e.g., displays, monitors, etc.), audio interfaces (e.g., microphones, speakers, etc.), and/or any other input/output component. The interface module 112 can include any interface for receiving information from a user, such as one or more tactile interfaces (e.g., keyboards, touch pads, etc.), audio interfaces (e.g., microphones, speakers, etc.), and/or any other input/output component.

The interface module 112 may include any interface for receiving information associated with a user and/or the user device 102 that may be used to track/monitor symptoms, activity, diet, vitals, and/or the like associated with a user of the user device 102. For example, the interface module 112 may include and/or be in communication with one or more devices/components associated with a user and/or the user device 102, such as an accelerometer, a pedometer, a geographical position sensing (GPC) module, an oximeter, a tactile sensor, and/or the like. The interface module 112 may receive and/or determine data/information (e.g., one or more signals, etc.) from the one or more devices/components associated with a user and/or the user device 102 may be analyzed (e.g., a long-term evaluation, etc.) to determine movement and impact velocities associated with the user device 102 and/or the user.

The interface module 112 can be and/or include any interface for presenting information to the user, such as screening questions and/or related symptomatic indicators. Screening questions may be associated with and/or include one or more symptomatic indicators. A symptomatic indicator may indicate a likelihood that the user (e.g., patient, subject, etc.) may be experiencing and/or be affected by a health issue, a medical issue, a physiological issue, a psychological issue, and/or the like. Screening questions may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. For example, screening questions may be designed to associate a military service history of a user with any somatic symptoms a user may be experiencing, pain/injury a user may be experiencing, illness a user may be experiencing, personal and/or social behavior associated with a user, depression symptoms a user may be experiencing, and/or the like to determine/identify correlations between certain symptoms and/or health/mental issues, such as a user's propensity for suicide. Screening questions may be associated with any topic, health/medical related issue, and/or the like relative to a user.

The interface module 112 can be and/or include any interface for receiving information from the user, such as responses to screening questions (and/or symptomatic indicators). The interface module 112 may be any interface for presenting and/or receiving any information to/from the user.

The user device 102 may include an analysis module 115. The analysis module 115 may determine data/information associated with screening questions and/or symptomatic indicators (e.g., responses to screening questions), such as a score that may indicate a health issue. For example, the analysis module 115 may determine, for each response (e.g., tactile response, audio response, patient provided response, etc.) to a screening questions and/or symptomatic indicators, a score (e.g., a score on a scale from 1-10, etc.). The analysis module 115 may access data/information associated with the screening questions and/or symptomatic indicators (e.g., stored in the memory 108 or any other location, etc.) and correlate each response to the screening questions and/or symptomatic indicators to the data/information associated with the screening questions and/or symptomatic indicators, such as an electronic medical record (EMR) associated with the user, a military service record associated with the user, and/or the like. By performing such correlations, the analysis module 115 may identify factors that may induce symptoms that affect a user and/or cause health/medical issues that affect the user that may be the result of particular occupational and/or other risk factors.

The analysis module 115 may determine scores to the screening questions. The analysis module 115 may, for example, determine scores to the screening questions by accessing data/information associated with the screening questions (and/or symptomatic indicators) and correlating each response to the screening questions (and/or symptomatic indicators) to the data/information associated with the screening questions (and/or symptomatic indicators), such as electronic medical record (EMR) associated with the user, a military service record associated with the user, previous responses to screening questions, and/or the like. In some instances, the analysis module 115 may determine scores to the screening questions by accessing data/information associated with the screening questions (and/or symptomatic indicators) and using machine learning to correlate each response to the screening questions (and/or symptomatic indicators) to the data/information associated with the screening questions (and/or symptomatic indicators).

The analysis module 115 may associate each screening questions and/or associated symptomatic indicator with a scale (e.g., a nominal scale, an ordinal scale, an interval scale, a ratio scale, etc.) of a plurality of scales. Each scale of the plurality of scales may a quantitative and/or a standard system for grading a response (or change from a previous response) and/or symptomatic indicator. The analysis module 115 may determine scores by determining, for each response (or change from a previous response) and/or symptomatic indicator, a scale. For example, the analysis module 115 may, for each response to the plurality of screening questions, based on an associated scale of the plurality of scales, may scale the response according to a screening question and/or symptomatic indicator. Each scaled response to a screening question may represent a score. The analysis module 115 may total (e.g., sum, average, means-square, etc.) scores derived from responses to determine an overall score associated with a user. The overall score may be associated with the health and/or wellbeing of the user. The overall score may indicate possible health and/or wellbeing issues. The user device 102 may display (via the interface module 112) a score and/or an overall score associated with a user. The user device 102 may determine scores based on any method.

The user device 102 may be in communication with the computing device 104. The computing device 104 may communicate with the user device 102 for providing data and/or services. The computing device 104 may allow the user device 102 to interact with remote resources such as data, devices, and files. The computing device may be configured as (or disposed at) a central location (e.g., a headend, or processing facility), which may receive content (e.g., data, input programming) from multiple sources. The computing device 104 may combine the content from the multiple sources and may distribute the content to user (e.g., subscriber) locations via a distribution system.

The computing device 104 may manage the communication between the user device 102 (and/or multiple user devices 102) and a database 130 for sending and receiving data therebetween. The database 130 may store a plurality of files (e.g., web pages), user identifiers or records, or other information. The user device 102 may request and/or retrieve a file from the database 130. The database 130 may store information relating to the user device 102 such as the address element 110 and/or the service element 112. The computing device 104 may obtain the device identifier 116 from the user device 102 and retrieve information from the database 130 such as the address element 118 and/or the service elements 120. The computing device 104 may obtain the address element 110 from the user device 102 and may retrieve the service element 120 from the database 130, or vice versa. Any information may be stored in and retrieved from the database 130. The database 130 may be disposed remotely from the computing device 104 and accessed via direct or indirect connection. The database 130 may be integrated with the computing system 104 or some other device or system. The database 130 may be and/or be associated with an electronic records system. The database 130 may include a user profile (e.g., user information, an electronic medical record (EMR), military service history, etc.) associated with a user and/or multiple users. Each user profile may include data/information such as a treatment and medical history associated with a user. A user profile may include the user's health (e.g., mental health, etc.) history and records catalogued in a standardized format. A user profile may be and/or include secure and/or encrypted data/information that may be searched, accessed, and/or queried to provide real-time information associated with health and/or medical decision-making. Scores derived from screening questions may be associated with a user profile.

In some instances, the computing device 104 (e.g., a cloud-based device, a server, an electronic records management device, etc.) may determine scores derived from responses to screening questions. For example, the user device 102 (via the interface module 112) may receive responses to screening questions and send the responses to the computing device 104. The user device 102 may send the responses to the computing device 104 via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The computing device 104 may receive the responses to the screening questions and determine scores based on the responses received.

The computing device 104 may access data/information stored in the database 130. The computing device 104 may access data/information associated with the plurality of symptomatic indicators, such as potential answers to screening questions, previous answers to screening questions, data/information from one or more devices/components associated with the user device 102, and/or the like. The computing device 104 may access data/information associated with the plurality of symptomatic indicators to determine the one or more scores. The computing device 104 may correlate each response to the screening questions (and/or symptomatic indicators) to the data/information associated with the screening questions (and/or symptomatic indicators), such as the medical and/or military record of a user, or data/signals from one or more devices associated with the user device 102 and/or the user. The computing device 104 may associate each screening questions and/or associated symptomatic indicator with a scale (e.g., a nominal scale, an ordinal scale, an interval scale, a ratio scale, etc.) of a plurality of scales. Each scale of the plurality of scales may a quantitative and/or a standard system for grading a response to a screening question and/or symptomatic indicator. The computing device 104 may determine scores by determining, for each response to a screening question and/or symptomatic indicator, a scale. The computing device 104 may, for each response to a screening question and/or symptomatic indicator, based on an associated scale of the plurality of scales, may scale the response according to a screening question and/or symptomatic indicator. Each scaled response to a screening question and/or symptomatic indicator may represent a score. The computing device 104 may total (e.g., sum, average, means-square, etc.) scores derived from responses to determine an overall score associated with a user. The overall score may be associated with the health and/or wellbeing of the user. The overall score may indicate possible health and/or wellbeing issues. The computing device 104 may determine scores by any method and may provide the scores to the user device 102.

The computing device 104 may provide the scores to the user device 102 via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The user device 102 may present, display, and/or cause display of the one or more scores. The user device 102 may present, display, and/or cause display of the one or more scores. The user device 102 may present, display, and/or cause display of data/information associated with the one or more scores, such as graphical, statistical, and/or any other analytical data/information associated with the one or more scores. For example, the user device 102 may present, display, and/or cause display of a graph that depicts user scores (and or responses to screening questions) over a time period and/or range.

The computing device 104 may store (e.g., via the database 130, etc.) scores. Storing scores may include associating the scores with user information (e.g., a user profile, an electronic medical record (EMR), a military service record, a user account, etc.) associated with the user. Storing the scores may include storing a score for each screening question and/or symptomatic indicator. Storing the scores may include storing additional data/information associated with the user, screening question, and/or the like.

One or more scores may be compiled with additional medical information associated with a symptomatic indicator. Specific diagnoses, and prescribed services and/or treatments may be determined based on a score. In some instances, the user device 102 may determine specific diagnoses, and prescribed services and/or treatments. For example, an application associated with the user device may determine specific diagnoses, and prescribed services and/or treatments based on responses to screening questions. In some instances, the computing device 104 may determine specific diagnoses, and prescribed services and/or treatments based on responses to screening questions received from the user device 102. I

In some cases, specific diagnoses and or a user profile may be modified, adjusted, updated, and/or the like based on additional information received and/or associated with a user profile. The user device 102 may solicit data/information associated with the user. For example, a user profile may be analyzed to determine a time (e.g., instance, date, period, etc.) when responses to screening questions and/or data/information from the interface 112 (e.g., one or more devices, an accelerometer, a pedometer, a geographical position sensing (GPC) module, an oximeter, a tactile sensor, etc.) associated with the user device 102 was received. The user device 102 (and/or a computing device 104) may determine, based on the time when the responses to the screening questions and/or the data/information from the one or more devices was received, a time frame, a time period, a time window, and/or the like. The time frame (e.g., time period, time window, etc.) may be relative to when additional responses to screening questions and/or additional data/information from the interface 112 (e.g., one or more devices, an accelerometer, a pedometer, a geographical position sensing (GPC) module, an oximeter, a tactile sensor, etc.) should be received, such as daily, weekly, monthly, and/or the like. In some instances, the time frame may be automatically determined based on the user profile and/or any symptomatic indicators determined to be affecting the user. In some instances, the time frame may be determined by the user. When additional responses to screening questions and/or additional data/information from the interface 112 (e.g., one or more devices, an accelerometer, a pedometer, a geographical position sensing (GPC) module, an oximeter, a tactile sensor, etc.) is not received within the time frame, the user device 102 (and/or computing device 104) may send a notification (e.g., a signal, a message, an email, a text, etc.) to the user device 102 and/or the user that causes the user to provide additional responses to screening questions and/or additional data/information from the interface 112.

The user device 102 (and/or computing device 104) may use the additional responses to screening questions and/or additional data/information from the interface 112 to update the user profile. The user profile and/o the updated user profile may be used to determine an alert condition. In some cases, an alert condition may be determined based on a score derived from a screening question. An alert condition may be and/or include an indication that a user is at risk for an emergency medical condition associated with a symptomatic indicator related to a screening question. Scores associated with a user profile may be compiled with additional medical information associated with each symptomatic indicator of a plurality of symptomatic indicators, and specific diagnosis, and prescribed services and/or treatments may be determined based on an alert condition.

Scores may be accessed and/or analyzed according to one or more rules associated screening question and/or associated symptomatic indicator. For example, a rule associated with a symptomatic indicator for pain a user may be experiencing may dictate that user scores that satisfy a threshold value may indicate an alert condition. A screening question may ask the user where they are experiencing pain, or use a symptomatic indicator (e.g., by displaying a 3D model) to enable the user to indicate where pain is experienced. The screening question may ask and/or a symptomatic indicator may probe a user to describe the type of pain (e.g., aching, burning, cramping, dull, earache, fatigue, gastrointestinal pain, headache, itching, sharp, stabbing, shocking, etc.) the user is experiencing. The user may provide a response based on or correlated to a scale from 1-10, where scores/values of 4 or greater are considered to be clinically significant pain, and therefore indicative of an alert condition. An alert condition may be determined based on any rule associated with associated screening question and/or associated symptomatic indicator. In some instances, the user device 102 (via the analysis module 115, etc.) may determine the alert condition. For example, an application associated with the user device 102 may determine the alert condition. In some instances, the computing device 104 may determine the alert condition based on the responses received from the user device 102.

The computing device 104 may include a machine learning and analysis module 138. In some instances, a machine learning and analysis module 138 may determine an alert condition and/or the likelihood that the user will be diagnosed with a health/medical issue related to a symptomatic indicator of the plurality of symptomatic indicators, based on a score for the symptomatic indicator. The machine learning and analysis module 138 may be and/or include a neural network.

In some cases, the machine learning and analysis module 138 may perform, in whole or in part, analytical function of the system 100. The machine learning and analysis module 138 may be configured to approximate the knowledge of a clinician/physician and a standard of mental, physical, and medical care by making discriminating judgments based on a probable cause of a mental, physical, and medical related diagnoses determined through the analysis of user health data (e.g., responses to screening questions, etc.) in view of a set or sets (e.g., a dataset and/or datasets, etc.) of clinical methodologies. The machine learning and analysis module 138 may use both fuzzy logic, Boolean models, and/or the like. Fuzzy logic, in contrast to more deterministic Boolean models, may provide analytical output of clinical/medical data sets in terms of clinical/medical probabilities rather than more rigid absolutes.

The machine learning and analysis module 138 may access and/or determine clinical probabilities, such as probable medical and/or health alert conditions. The machine learning and analysis module 138 may include contemporaneous determined and stored user health data (e.g., from screening questions, from one or more devices, etc.). The machine learning and analysis module 138 may comprise a collection of clinical data/information, such as historical symptoms, diagnoses and outcomes, along with time development of health/medical issues. The machine learning and analysis module 138 may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. For example, the machine learning and analysis module 138 may associate a military service history of a user with any somatic symptoms a user may be experiencing, pain/injury a user may be experiencing, illness a user may be experiencing, personal and/or social behavior associated with a user, depression symptoms a user may be experiencing, and/or the like to determine/identify correlations between certain symptoms and/or health/mental issues, such as a user's propensity for suicide. The machine learning and analysis module 138 may analyze data/information associated with any topic, health/medical related issue, and/or the like relative to a user. Data/information, such as responses derived from screening questions, and/or one or more signals (e.g., additional data/information) collected from one or more devices associated with the user device 102 (e.g., interface module 112, an accelerometer, a pedometer, a geographical position sensing (GPC) module, an oximeter, a tactile sensor. etc.), may be coded and/or encoded and input into the machine learning and analysis module 138 to populate and/or train the computing device 104 with clinical data/information. The machine learning and analysis module 138 may use the clinical data/information to determine, derive, and/or predict clinical, medical, health, and diagnostic outcomes, such as one or more alerts conditions.

Scores derived from responses to the screening questions may be used to create a dataset. The machine learning and analysis module 138 may compare the dataset to previously stored datasets and/or information. Comparing the dataset to stored datasets and/or information may provide an indication of a possible diagnosis of a health/medical issue associated with a symptomatic indicator of the plurality of symptomatic indicators. In some instances, scores derived from responses to the screening questions from multiple users and/or multiple user devices 102 may be used to create a training dataset. The training dataset may be used to train the machine learning and analysis module 138. When new data/information (e.g., clinical information, responses to screening questions, training datasets, etc.) is provided to the machine learning and analysis module 138, the machine learning and analysis module 138 may update any stored data/information and adapt to any changing parameters (e.g., changes to a dataset, etc.) associated with the clinical data/information. The machine learning and analysis module 138 may verify conclusions, diagnoses, and/or the like for accuracy and/or significance. The machine learning and analysis module 138 may store test cases, appropriate outcomes and the relative occurrence of misidentification of the proper outcome and/or diagnosis. The machine learning and analysis module 138 may establish a threshold of acceptable misidentifications or misdiagnoses.

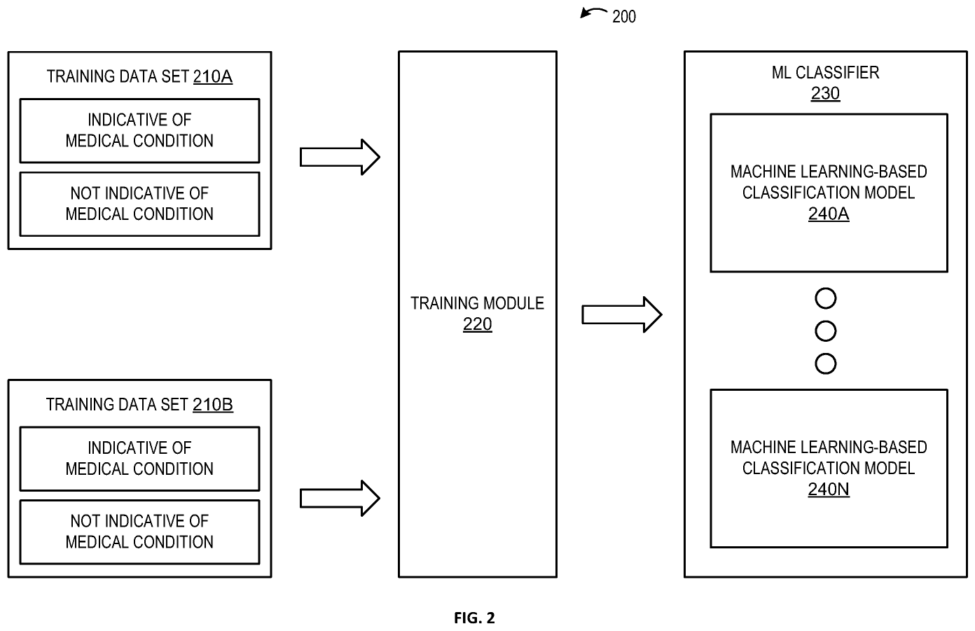

The machine learning and analysis module 138 may use the clinical data/information to determine, derive, and/or predict clinical, medical, health, and diagnostic outcomes (referred to herein as “medical conditions”). Turning now to FIG. 2, an example system 200 depicting an implementation of the machine learning and analysis module 138 is shown. The system 200 may be configured to use machine learning techniques to train, based on an analysis of one or more training data sets 210A-210B by a training module 220, at least one machine learning-based classifier 230. The machine learning-based classifier 230 may be the machine learning and analysis module 138 described herein. The machine learning-based classifier 230 may be configured to determine whether patient data (e.g., user data) for a particular patient/user is indicative or not indicative of a particular medical condition. Patient data for a particular patient/user and/or training data may comprise one or more pieces of clinical data/information and/or stored user health data as described herein. The training data set 210A may comprise labeled patient data for a first plurality of patients/users (e.g., labeled as being indicative (or not) of a presence of particular medical condition). The training data set 210B (e.g., a second portion of the second core data) may also comprise labeled patient data for a second plurality of patients/users (e.g., labeled as being indicative (or not) of a presence of particular medical condition). The labels may comprise “Indicative of Medical Condition” and “Not Indicative of Medical Condition.”

Patient data used for training may be randomly assigned to the training data set 210B or to a testing data set. In some implementations, the assignment of data to a training data set or a testing data set may not be completely random. In this case, one or more criteria may be used during the assignment, such as ensuring that similar numbers of patient/user datasets with different labels and different medical conditions are in each of the training and testing data sets. In general, any suitable method may be used to assign the data to the training or testing data sets, while ensuring that the distributions of “Indicative of Medical Condition” and “Not Indicative of Medical Condition” labels are somewhat similar in the training data set and the testing data set.

The training module 220 may train the machine learning-based classifier 230 by extracting a feature set from a first portion of the training data set 210A according to one or more feature selection techniques. The term “feature” as used herein may refer to one or more pieces of patient data, such as one or more pieces of clinical data/information and/or stored user health data as described herein. The training module 220 may further define the feature set obtained from the training data set 210A by applying one or more feature selection techniques to a second portion of the training data set 210B that includes statistically significant features of positive examples (e.g., one or more pieces of patient data labeled as “Indicative of Medical Condition”) and statistically significant features of negative examples (e.g., one or more pieces of patient data labeled as “Not Indicative of Medical Condition”).

The training module 220 may extract a feature set from the training data set 210A and/or the training data set 210B in a variety of ways. The training module 220 may perform feature extraction multiple times, each time using a different feature-extraction technique. In an embodiment, the feature sets generated using the different techniques may each be used to generate different machine learning-based classification models 240. For example, the feature set with the highest quality metrics may be selected for use in training. The training module 220 may use the feature set(s) to build one or more machine learning-based classification models 240A-240N that are configured to indicate whether or not one or more pieces of patient data for a patient/user are associated with indicative or not indicative of a particular medical condition.

The training data set 210A and/or the training data set 210B may be analyzed to determine any dependencies, associations, and/or correlations between extracted features and the indicative/not indicative labels in the training data set 210A and/or the training data set 210B. The identified correlations may have the form of a list of features that are associated with each label. The features may be considered as variables in the machine learning context. The term “feature,” as used herein, may refer to any characteristic of an item of data that may be used to determine whether the item of data falls within one or more specific categories. By way of example, the features described herein may comprise one or more pieces of patient data.

A feature selection technique may comprise one or more feature selection rules. The one or more feature selection rules may comprise a “data point occurrence rule” associated with one or more pieces of patient data. The data point occurrence rule may comprise determining which pieces of patient data in the training data set 210A occur over a threshold number of times and identifying those pieces of patient data that satisfy the threshold as candidate features. For example, any piece of patient data that appears greater than or equal to 2 times in the training data set 210A may be considered as candidate features. Any piece of patient data appearing less than 2 times may be excluded from consideration as a feature. Any threshold amount may be used as needed.

A single feature selection rule may be applied to select features or multiple feature selection rules may be applied to select features. The feature selection rules may be applied in a cascading fashion, with the feature selection rules being applied in a specific order and applied to the results of the previous rule. For example, the clinical data point occurrence rule may be applied to the training data set 210A to generate a first list of imaging result attributes. A final list of candidate features may be analyzed according to additional feature selection techniques to determine one or more candidate groups (e.g., pieces/groups of patient data that may be used to predict whether new patient/user data is indicative or not of a particular medical condition). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., indicative or not of a particular medical condition).

As another example, one or more candidate feature groups may be selected according to a wrapper method. A wrapper method may be configured to use a subset of features and train the machine learning-based classifier 230 using the subset of features. Based on the inferences that drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature groups. Forward feature selection is an iterative method that begins with no features in the training module 220. In each iteration, the feature which best improves the machine learning-based classifier 230 is added until an addition of a new feature does not improve the performance of the machine learning-based classifier 230. In an embodiment, backward elimination may be used to identify one or more candidate feature groups. Backward elimination is an iterative method that begins with all features in the machine learning-based classifier 230. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 220 has generated a feature set(s), the training module 220 may generate one or more machine learning-based classification models 240A-240N based on the feature set(s). For example, the training module 220 may use the feature sets extracted from the training data set 210A and/or the training data set 210B to build a machine learning-based classification model 240A-240N for each classification category (e.g., indicative/not indicative) for each particular medical condition. In some examples, the machine learning-based classification models 240A-340N may be combined into a single machine learning-based classification model 240. Similarly, the machine learning-based classifier 230 may represent a single classifier containing a single or a plurality of machine learning-based classification models 240 and/or multiple classifiers containing a single or a plurality of machine learning-based classification models 240.

The extracted features may be combined in the machine learning-based classification models 240A-240N using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 230 may comprise one or more decision rules or mappings for each feature and for each particular medical condition to assign new patient data to a class (e.g., indicative/not indicative of the particular medical condition).

The one or more decision rules or mappings for each particular medical condition and the machine learning-based classifier 230 may be used to predict a label (e.g., indicative/not indicative of the particular medical condition) for new patient data for a plurality of new patients/users (e.g., unlabeled patient data) in the testing data set. In one example, the prediction for each of the plurality of new patients/users in the testing data set includes a confidence level that corresponds to a likelihood or a probability that the corresponding new patient data for the particular new patient/user belongs in the predicted indicative/not indicative of the particular medical condition status. The confidence level may be a value between zero and one, and it may represent a likelihood that the corresponding new patient data for the particular new patient/user belongs to a particular indicative/not indicative of the particular medical condition status. In one example, when there are two statuses (e.g., indicative/not indicative of the particular medical condition), the confidence level may correspond to a value p, which refers to a likelihood that corresponding new patient data for the particular new patient/user belongs to the first status (e.g., indicative of the particular medical condition). In this case, the value 1−p may refer to a likelihood that the corresponding new patient data for the particular new patient/user belongs to the second status (e.g., not indicative of the particular medical condition). In general, multiple confidence levels may be provided for each of the plurality of new patients/users and for each feature. As noted above, the term “feature” as used herein may refer to one or more pieces of patient data, such as one or more pieces of clinical data/information and/or stored user health data as described herein. A top performing feature—or a group of top performing features—may be determined by comparing the result obtained for each of the plurality of new patients/users with a known indicative/not indicative of the particular medical condition status for each in the testing data set. In general, the top performing candidate feature—or group of top performing candidate features—will have results that closely match the known indicative/not indicative of the particular medical condition status.

Once trained, the machine learning-based classifier 230 may determine which pieces of new patient data, such as one or more pieces of clinical data/information and/or stored user health data, are most indicative of a particular medical condition. For example, as further described below, new patient data associated with a new patient/user may be received. The new patient data may be provided to the machine learning-based classifier 230. The machine learning-based classifier 230 may, based on the top performing candidate feature—or group of top performing candidate features, classify the new patient data as being indicative or not indicative of a particular medical condition.

Returning to FIG. 1, when an alert condition (and/or any other condition associated with the plurality of screening questions) is determined, such as based on a score for a symptomatic indicator of the plurality of symptomatic indicators, the clinical device 107 and/or an associated clinician may be determined. The computing device 104 may use information associated with the user device 102, such as the device identifier 116 and/or an identifier associated with the user to determine the clinical device 107 and/or an associated clinician. The computing device 104 may use the device identifier 116 and/or an identifier associated with the user to determine the user information (e.g., user profile, electronic medical record (EMR), military service record, etc.).

The user information may indicate one or more clinical devices 107 and/or clinicians associated with the user and/or user device 102. A clinical devices 107 and/or clinician be associated with various screening question and/or symptomatic indicators. In some instances, the clinical devices 107 and/or clinician may be determined based on a score derived from screening questions for a particular symptomatic indicator. The clinical devices 107 and/or clinician may be associated with a respective symptomatic indicator (or health or medical issue related to the symptomatic indicator) based on a clinical ability of a clinician associated with a clinical device 107 to respond to, treat, diagnose, provide care for, and/or otherwise address any issue relating to and/or associated with the symptomatic indicator. The clinical devices 107 and/or clinician may each be ranked based on a clinical ability of a clinician associated with a clinical device 107 to respond to, treat, diagnose, provide care for, and/or otherwise address any issue relating to and/or associated with a symptomatic indicator. The computing device 104 (or user device 102) may use the rank associated with the clinical device 107 and/or an associated clinician to determine the clinical device 107 and/or an associated clinician.

The computing device 104 (or user device 102) may send a notification (e.g., a signal, a message, an email, a text, etc.) to the clinical device 107 based on determining an alert condition, medical diagnosis, predictive diagnosis, and/or any other related information associated with a user of the user device 102. The computing device 104 (or user device 102) may send the notification to the clinical device 107 via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The notification may be sent to the clinical device 107 via an application. For example, the notification may be sent to the clinical device 107 via an application program interface (API) associated with the user device 102, the computing device 104, the clinical device 107, and/or the like.

The clinical device 107 may include an interface module 139. The interface module 139 may include and/or be associated with a communication interface such as a web browser (e.g., Internet Explorer, Mozilla Firefox, Google Chrome, Safari, or the like). Other software, hardware, and/or interfaces can be used to provide communication between the clinician and one or more of the clinical device 107, the computing device 104, the user device 102, and/or any other device. The interface module 139 can request or query various files from a local source and/or a remote source, such as data/information associated with and/or including screening questions and/or symptomatic indicators. The interface module 139 may provide an interface for a clinician to interact with the clinical device 107, the computing device 104, the user device 102, and/or any other device. The interface module 139 can include any interface for presenting information to a clinician, such as one or more visual interfaces (e.g., displays, monitors, etc.), audio interfaces (e.g., microphones, speakers, etc.), and/or any other input/output component. The interface module 139 can include any interface for receiving information from a clinician/user, such as one or more tactile interfaces (e.g., keyboards, touch pads, etc.), audio interfaces (e.g., microphones, speakers, etc.), and/or any other input/output component. The interface module 139 can be and/or include any interface for presenting information to the clinician/user, such as scores from screening questions, predictive diagnoses of medical and/or health issues, and/or any data/information.

The interface module 139 may enable a clinician/user to view information about a notification and/or alert condition, such as information about a user/patient status, physiological parameter values, trend data, audio/video of the user/patient, combinations of the same, or the like. The notification may cause the clinical device 107 to perform an action. For example, the notification may cause the clinical device to schedule an appointment (e.g., via an appointment scheduling system, online/web-based system, etc.) to meet with the clinician and/or a clinical staff associated with the clinical device 107. The interface module 139 may provide functionality for a clinician/user to respond to a notification, annotate an alarm/diagnosis, indicate that the clinician can or cannot respond to the notification, schedule an appointment for medical, mental, and/or health care/treatment, and/or the like.

In some cases, the user device 102 and/or the computing device 104 may analyze data/information associated with a plurality of users. For example, the machine learning and analysis module 138 (e.g., the machine learning-based classifier 230) and/or the analysis module 115 may analyze data/information associated with user information (e.g., a user profile, an electronic medical record (EMR), a military service record, a user account, etc.) from and/or associated with multiple users. The machine learning and analysis module 138 (e.g., the machine learning-based classifier 230) and/or the analysis module 115 may analyze data/information associated with a plurality of user profiles associated with a plurality of users to determine and/or identify one or more target areas (e.g., geographical areas) where individuals likely may be associated with one or more symptomatic indicators.

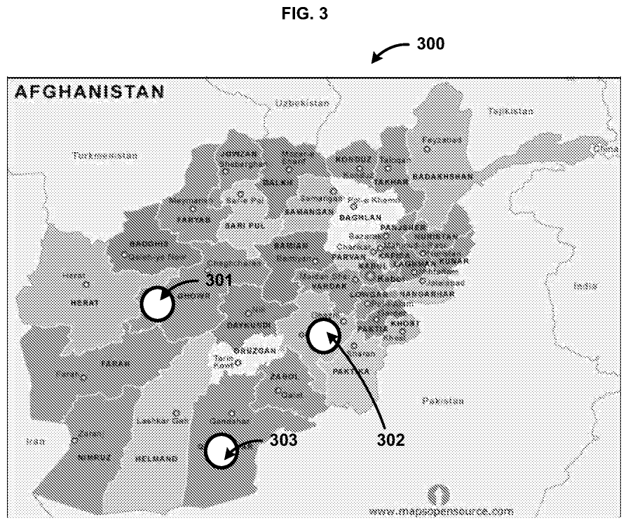

FIG. 3 is an example a screenshot 300 of graphical user interface (GUI) generated by the user device 102. The machine learning and analysis module 138 (e.g., the machine learning-based classifier 230) and/or the analysis module 115 may predict and/or determine target areas (e.g., geographical areas) 301, 302, and 303 where there may a statistically relevant occurrence of a particular symptom (symptomatic indicator) and/or health related issue. For example, the target area 301 may have a high rate of suicide. The target area 302 or target area 303 may have a statistically high occurrence of people with cancer, blindness, deafness, or any other occupational or regional induced symptom and/or health issue. The machine learning and analysis module 138 (e.g., the machine learning-based classifier 230) and/or the analysis module 115 may use user demographics, responses to screening questions, and any other related data/information to determine, for particular symptom (symptomatic indicator) and/or health related issue, commonalities among users. Commonalities among users may be used to determine why a particular symptom (symptomatic indicator) and/or health related issue is occurring in a given target area. Target areas may identify any occurrence of a health, medical, or mental issue, as well as reasons for such occurrences. For example, the machine learning and analysis module 138 (e.g., the machine learning-based classifier 230) and/or the analysis module 115 may determine the target area 301. The target area 301 may be a geographical area where individuals experience a high rate of suicide. By correlating multiple user profile from users that may have worked, been stationed in, or otherwise been exposed to the target area 301, the machine learning and analysis module 138 (e.g., the machine learning-based classifier 230) and/or the analysis module 115 may determine why the target area 301 has such a high suicide rate for such users. For example, responses to screening questions provided from users that may have worked, been stationed in, or otherwise been exposed to the target area 301 may indicate that many post-traumatic stress inducing events occurred in the target area 301 and thus results in a high suicide rate for the target area 301.

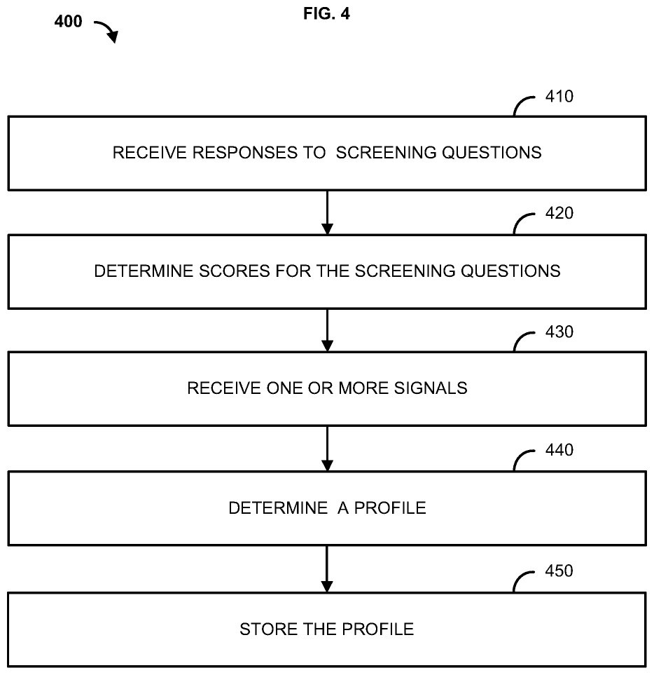

FIG. 4 shows a flowchart of an example method 400 for comprehensive symptom analysis. To aid in the provision of health care to users (e.g., veterans, health care participants, etc.), at 410, a plurality of responses to a plurality of screening questions may be received. A user device (e.g., the user device 102, a mobile device, a smart device, computing device, etc.) may receive a plurality of responses to a plurality of screening questions. The user device may present, display, and/or cause display of the plurality of screening questions. For example, the user device may include and/or be associated with a display for presenting/displaying a plurality of screening questions. In some instances, the user device may be configured with an application that causes a plurality of screening questions to be presented and/or displayed. The plurality of screening questions may include questions associated with a plurality of symptomatic indicators associated with health and wellbeing. The screening questions may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. Symptomatic indicators may include any information, response, and/or the like indicative of a health, medical, and/or mental issue, such as somatic symptoms a user may be experiencing, pain/injury a user may be experiencing, illness a user may be experiencing, personal and/or social behavior of the user, depression symptoms the user may be experiencing, and/or the like. The plurality of symptomatic indicators may include symptomatic indicators and/or information associated with any health and/or wellbeing issue. For example, a screening question for and/or associated with a symptomatic indicator for depression and/or a related mental issue may include, “has your military service changed the way you view your world?” As another example, a screening question for and/or associated with a symptomatic indicator for various behavior issues may include, “If a friend called to ask you to come over for dinner, how are you most likely to respond?” The user device may present, display, and/or cause display of any question, inquiry, and/or the like associated with user health and wellbeing. The user device may present, display, and/or cause display of any question, inquiry, and/or the like.

The user device may receive responses to the screening questions. The responses to the screening questions may include tactile responses, audio responses, and/or any other response associated with a user. For example, in some cases, the user device may present, display, and/or cause display of a three-dimensional human model and present questions to the user such as “where do you feel pain?” The user may respond to the question by rotating the three-dimensional human model to indicate an area and/or region where the user is experiencing pain. The user device may present, display, and/or cause display of a prompt that ask the user to “describe what you feel.” The user may respond by manually entering a response, or accessing and/or selecting one or more provided responses to the prompt that ask the user to “describe what you feel,” that allow the user to indicate that they feel aching, burning, cramping, dull, earache, fatigue, gastrointestinal pain, headache, itching, sharp, stabbing, shocking, and/or the like. The user may remove, or cause to remove, both section and partial section views of the three-dimensional human model, allowing the user to more accurately indicate both depth and travel pattern of pain and/or sensations.

At 420, one or more scores may be determined. A score (e.g., a dichotomous score, a polytomous score, etc.) may be determined for each symptomatic indicator of the plurality of symptomatic indicators, based on the response to each of the plurality of screening questions. Each symptomatic indicator of the plurality of symptomatic indicators may be associated with a scale (e.g., a nominal scale, an ordinal scale, an interval scale, a ratio scale, etc.) of a plurality of scales. Each scale of the plurality of scales may a quantitative and/or a standard system for grading a symptomatic indicator of the plurality of symptomatic indicators. The one or more scores may be determined by determining, for each symptomatic indicator of the plurality of symptomatic indicators, a scale of the plurality of scales. Each response to the plurality of screening questions, based on an associated scale of the plurality of scales, may be scaled. Each scaled response to the plurality of screening questions may represent a score of the one or more scores. In some instances, each score of the one or more scores may be totaled (e.g., summed, averaged, means-squared, etc.) to determine an overall score associated with the user. The overall score may be associated with the health and/or wellbeing of the user. The overall score may indicate possible health and/or wellbeing issues.

In some instances, the user device may determine the one or more scores. For instance, an application associated with and/or installed on the user device may determine the one or more scores based on the responses to the plurality of screening questions. The user device may determine the one or more scores based on any method. In some instances, a computing device (e.g., cloud-based device, server, electronic records management device, etc.) may determine the one or more scores. For example, the user device may receive the responses to the plurality of screening questions and send the responses to the computing device. The user device may send the responses to the computing device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The computing device may receive the responses to the plurality of screening questions and determine the one or more scores. The computing device may access data/information associated with the plurality of symptomatic indicators to determine the one or more scores. The computing device may determine the one or more scores by any method and may provide the one or more scores to the user device. The computing device may provide the one or more scores to the user device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique.

At 430, one or more signals (e.g., data/information, etc.) that track symptoms, activity, diet, and/or vitals of the user may be received. The user device may receive the one or more signals (e.g., data/information, etc.) from an interface (e.g., interface 112, etc.) associated with the user device. The interface may be, comprise, and/or be associated with one or more devices, such as an accelerometer, a pedometer, a geographical position sensing (GPC) module, an oximeter, a tactile sensor, and/or the like.

In some instances, the user device 102 may solicit data/information associated with the user. For example, the user device may determine a time when previous responses to screening questions and/or data/information from the interface associated with the user device was received. The user device may determine, based on the time when the previous responses to the screening questions and/or the data/information from the one or more devices was received, a time frame, a time period, a time window, and/or the like. The time frame (e.g., time period, time window, etc.) may be relative to when additional responses to screening questions and/or additional data/information from the interface associated with the user device should be received, such as daily, weekly, monthly, and/or the like. In some instances, the time frame may be automatically determined based on the user profile and/or any symptomatic indicators determined to be affecting the user. In some instances, the time frame may be determined by the user. When additional responses to screening questions and/or additional data/information from the interface (e.g., one or more devices, an accelerometer, a pedometer, a geographical position sensing (GPC) module, an oximeter, a tactile sensor, etc.) is not received within the time frame, the user device may send a notification (e.g., a signal, a message, an email, a text, etc.) to the user device and/or the user that causes the user to provide additional responses to screening questions and/or additional data/information from the interface.

At 440, a user profile may be generated. The user profile may be generated by the user device. The user profile may include the score for each symptomatic indicator of the plurality of symptomatic indicators and the one or more signals. The user profile may associate the one or more scores with the one or more signals. The user profile may include at least the score for each symptomatic indicator of the plurality of symptomatic indicators. The user profile may include additional information related to the user, such as an electronic medical record, or military service record associated with the user.

The user profile may include any indication of a possible diagnosis of a health/medical issue associated with a symptomatic indicator of the plurality of symptomatic indicators. For example, the score for each symptomatic indicator of the plurality of symptomatic indicators and the one or more signals may be used to create a dataset. The dataset may be compared to datasets and/or information stored by a machine learning module (e.g., the machine learning and analysis module 138/the machine learning-based classifier 230). Comparing the dataset to datasets and/or information stored by the machine learning module machine learning module may provide an indication of a possible diagnosis of a health/medical issue associated with a symptomatic indicator of the plurality of symptomatic indicators.

In some instances, scores derived from responses to the screening questions from multiple users and/or user devices may be used to create a training dataset. The training dataset may be used to train the machine learning module. When new data/information (e.g., clinical information, responses to screening questions, training datasets, etc.) is provided to the machine learning module, the machine learning module may update any stored data/information and adapt to any changing parameters (e.g., changes to a dataset, etc.) associated with the clinical data/information. The machine learning module may verify conclusions, diagnoses, and/or the like for accuracy and/or significance. The machine learning module may store test cases, appropriate outcomes and the relative occurrence of misidentification of the proper outcome and/or diagnosis. The machine learning module may establish a threshold of acceptable misidentifications or misdiagnoses.

At 450, the user profile may be stored. In some instances, the user device may store the user profile. In some instances a computing device (e.g., the computing device 104, server, etc.) may store the user profile. The user device may send the user profile (and/or data/information associated with the user profile) to the computing device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The computing device may receive a device identifier of the user device and a user identifier of the user. The computing device may use the device identifier of the user device and/or the user identifier of the user to authenticate the user device and/or user prior to storing the user profile.

In some instances, the user profile may be used to determine an alert condition. An alert condition may be determined based on a score associated for a symptomatic indicator of the plurality of symptomatic indicators. An alert condition may be and/or include an indication that the user is at risk for an emergency medical condition associated with a symptomatic indicator of the plurality of symptomatic indicators. The one or more scores may be compiled with additional medical information associated with each symptomatic indicator of the plurality of symptomatic indicators, and specific diagnosis, and prescribed services and/or treatments may be determined based on an alert condition. In some instances, the user device may determine the alert condition. For example, an application associated with the user device may determine the alert condition. In some instances, the computing device (and/or a device/system in communication with the computing device, etc.) may determine the alert condition based on the responses received from the user device.

To determine an alert condition, each score of the one or more scores may be accessed and/or analyzed according to one or more rules associated with each symptomatic indicator of the plurality of symptomatic indicators. For example, a rule associated with a symptomatic indicator for pain intensity may dictate that user scores that satisfy a threshold value may indicate an alert condition. Such that a score/value of 4 or greater (on an associated scale ranging from values 1-10) are considered to be clinically significant pain, and therefore indicative of an alert condition. An alert condition may be determined based on any rule associated with each symptomatic indicator of the plurality of symptomatic indicators.

In some instances, an alert condition may be determined by a machine learning module (e.g., the machine learning and analysis module 138/the machine learning-based classifier 230). For example, to determine a likelihood that a user will have a diagnosis of a health/medical issue associated with a symptomatic indicator of the plurality of symptomatic indicators, based on a score for the symptomatic indicator. The computing device and/or the user device may be in communication with (and/or comprise) a machine learning module (e.g., the machine learning and analysis module 138/the machine learning-based classifier 230). The machine learning module may include contemporaneous determined and stored user health data (e.g., from screening questions, the user profile, etc.). The machine learning module may comprise a collection of clinical data/information, such as historical symptoms, diagnoses and outcomes, along with time development of health/medical issues and symptomatic indicators. The clinical data/information may be coded and/or encoded and input into the machine learning module to populate and/or train the network with clinical data/information that may be used to determine, derived, and/or predict clinical, medical, health, and diagnostic outcomes, such as one or more alerts conditions. When new data/information (e.g., clinical information, responses to screening questions, etc.) is provided to the machine learning module, the machine learning module may update any stored data/information and adapt to any changing parameters associated with the clinical data/information. The machine learning module may verify conclusions, diagnoses, and/or the like for accuracy and/or significance. The machine learning module may store test cases, appropriate outcomes and the relative occurrence of misidentification of the proper outcome and/or diagnosis. The machine learning module may establish a threshold of acceptable misidentifications or misdiagnoses.

In some cases, when an alert condition (and/or any other condition associated with the plurality of screening questions) is determined, such as based on a score for a symptomatic indicator of the plurality of symptomatic indicators, a clinical device (e.g., the clinical device 107, a server, etc.) and/or an associated clinician may be determined. In some cases, the user device, based on determining the alert condition, may determine the clinical device and/or an associated clinician. For example, an application associated with and/or installed on the user device, based on determining the alert condition, may determine the clinical device and/or an associated clinician. In some cases, the computing device, based on determining the alert condition, may determine the clinical device and/or an associated clinician.

In some instances, the device identifier associated with the user device and/or the identifier associated with the user may be used to determine the clinical device and/or an associated clinician. For example, the device identifier associated with the user device and/or the identifier associated with the user may be used to determine the user profile. The user profile may indicate one or more clinical devices and/or clinicians associated with the user and/or user device. The one or more clinical devices and/or clinicians may each be associated with a symptomatic indicator (or one or more symptomatic indicators) of the plurality of symptomatic indicators. In some instances, the one or more clinical devices and/or clinicians may be determined based on a symptomatic indicator of the plurality of symptomatic indicators, such as based on a score derived from the plurality of medical questions for a particular symptomatic indicator of the plurality of symptomatic indicators. For example, each symptomatic indicator of the plurality of symptomatic indicators may be associated with one or more clinical devices and/or clinicians. The one or more one or more clinical devices and/or clinicians may be associated with a respective symptomatic indicator of the plurality of symptomatic indicators based on a clinical ability of a clinician associated with a clinical device to respond to, treat, diagnose, provide care for, and/or otherwise address any issue relating to and/or associated with the symptomatic indicator. The one or more clinical devices and/or clinicians may each be ranked based on a clinical ability of a clinician associated with a clinical device to respond to, treat, diagnose, provide care for, and/or otherwise address any issue relating to and/or associated with the symptomatic indicator. The rank associated with the clinical device and/or an associated clinician may be used to determine the clinical device and/or an associated clinician.

In some instances, the clinical device (e.g., the clinical device 107, a server, etc.) and/or an associated clinician may receive a notification (e.g., a signal, a message, an email, a text, etc.). In some cases, the user device, based on determining the alert condition, may send the notification to the clinical device. For example, an application associated with and/or installed on the user device, based on determining the alert condition, may send the notification to the clinical device. In some cases, the computing device, based on determining the alert condition, may send the notification to the clinical device. The notification may be sent to the clinical device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The notification may be sent to the clinical device via an application associated with and/or installed on the user device, associated with the computing device, and/or the like. For example, the notification may be sent to the clinical device via an application program interface (API) associated with the user device, the computing device, the clinical device, and/or the like.

The notification may cause the clinical device to perform an action. For example, the notification may cause the clinical device to schedule an appointment (e.g., via an appointment scheduling system, online/web-based system, etc.) to meet with the clinician and/or a clinical staff associated with the clinical device. The notification may cause the clinical device to send data/information associated with the user of the user device to the user device and/or computing device. The data/information associated with the user of the user device may include appointment reminders, health and/or medical advice/instructions, information relating to a symptomatic indicator of the plurality of symptomatic indicators, and/or the like. The data/information associated with the user may include any data/information.

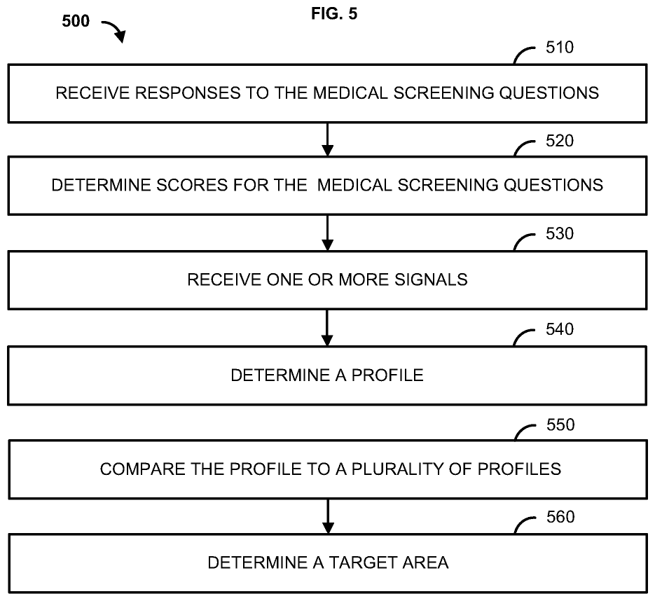

FIG. 5 shows a flowchart of an example method 500 for comprehensive symptom analysis. To aid in the provision of health care to users (e.g., veterans, health care participants, etc.), at 510, a plurality of responses to a plurality of screening questions may be received. A user device (e.g., the user device 102, a mobile device, a smart device, computing device, etc.) may receive a plurality of responses to a plurality of screening questions. The user device may present, display, and/or cause display of the plurality of screening questions. For example, the user device may include and/or be associated with a display for presenting/displaying a plurality of screening questions. In some instances, the user device may be configured with an application that causes a plurality of screening questions to be presented and/or displayed. The plurality of screening questions may include questions associated with a plurality of symptomatic indicators associated with health and wellbeing. The screening questions may place emphasis on collecting information on early diagnosis and/or under-diagnosis of long-term health effects due to occupational and/or regional exposure to various risk factors, such as exposure to chemicals, high decibel sound, post-traumatic stress disorder (PTSD) inducing events, and/or the like. Symptomatic indicators may include any information, response, and/or the like indicative of a health, medical, and/or mental issue, such as somatic symptoms a user may be experiencing, pain/injury a user may be experiencing, illness a user may be experiencing, personal and/or social behavior of the user, depression symptoms the user may be experiencing, and/or the like. The plurality of symptomatic indicators may include symptomatic indicators and/or information associated with any health and/or wellbeing issue. For example, a screening question for and/or associated with a symptomatic indicator for depression and/or a related mental issue may include, “has your military service changed the way you view your world?” As another example, a screening question for and/or associated with a symptomatic indicator for various behavior issues may include, “If a friend called to ask you to come over for dinner, how are you most likely to respond?” The user device may present, display, and/or cause display of any question, inquiry, and/or the like associated with user health and wellbeing. The user device may present, display, and/or cause display of any question, inquiry, and/or the like.

The user device may receive responses to the screening questions. The responses to the screening questions may include tactile responses, audio responses, and/or any other response associated with a user. For example, in some cases, the user device may present, display, and/or cause display of a three-dimensional human model and present questions to the user such as “where do you feel pain?” The user may respond to the question by rotating the three-dimensional human model to indicate an area and/or region where the user is experiencing pain. The user device may present, display, and/or cause display of a prompt that ask the user to “describe what you feel.” The user may respond by manually entering a response, or accessing and/or selecting one or more provided responses to the prompt that ask the user to “describe what you feel,” that allow the user to indicate that they feel aching, burning, cramping, dull, earache, fatigue, gastrointestinal pain, headache, itching, sharp, stabbing, shocking, and/or the like. The user may remove, or cause to remove, both section and partial section views of the three-dimensional human model, allowing the user to more accurately indicate both depth and travel pattern of pain and/or sensations.

At 520, one or more scores may be determined. A score (e.g., a dichotomous score, a polytomous score, etc.) may be determined for each symptomatic indicator of the plurality of symptomatic indicators, based on the response to each of the plurality of screening questions. Each symptomatic indicator of the plurality of symptomatic indicators may be associated with a scale (e.g., a nominal scale, an ordinal scale, an interval scale, a ratio scale, etc.) of a plurality of scales. Each scale of the plurality of scales may a quantitative and/or a standard system for grading a symptomatic indicator of the plurality of symptomatic indicators. The one or more scores may be determined by determining, for each symptomatic indicator of the plurality of symptomatic indicators, a scale of the plurality of scales. Each response to the plurality of screening questions, based on an associated scale of the plurality of scales, may be scaled. Each scaled response to the plurality of screening questions may represent a score of the one or more scores. In some instances, each score of the one or more scores may be totaled (e.g., summed, averaged, means-squared, etc.) to determine an overall score associated with the user. The overall score may be associated with the health and/or wellbeing of the user. The overall score may indicate possible health and/or wellbeing issues.

In some instances, the user device may determine the one or more scores. For instance, an application associated with and/or installed on the user device may determine the one or more scores based on the responses to the plurality of screening questions. The user device may determine the one or more scores based on any method. In some instances, a computing device (e.g., cloud-based device, server, electronic records management device, etc.) may determine the one or more scores. For example, the user device may receive the responses to the plurality of screening questions and send the responses to the computing device. The user device may send the responses to the computing device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The computing device may receive the responses to the plurality of screening questions and determine the one or more scores. The computing device may access data/information associated with the plurality of symptomatic indicators to determine the one or more scores. The computing device may determine the one or more scores by any method and may provide the one or more scores to the user device. The computing device may provide the one or more scores to the user device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique.

At 530, one or more signals (e.g., data/information, etc.) that track symptoms, activity, diet, and/or vitals of the user may be received. The user device may receive the one or more signals (e.g., data/information, etc.) from an interface (e.g., interface 112, etc.) associated with the user device. The interface may be, comprise, and/or be associated with one or more devices, such as an accelerometer, a pedometer, a geographical position sensing (GPC) module, an oximeter, a tactile sensor, and/or the like.

In some instances, the user device 102 may solicit data/information associated with the user. For example, the user device may determine a time when previous responses to screening questions and/or data/information from the interface associated with the user device was received. The user device may determine, based on the time when the previous responses to the screening questions and/or the data/information from the one or more devices was received, a time frame, a time period, a time window, and/or the like. The time frame (e.g., time period, time window, etc.) may be relative to when additional responses to screening questions and/or additional data/information from the interface associated with the user device should be received, such as daily, weekly, monthly, and/or the like. In some instances, the time frame may be automatically determined based on the user profile and/or any symptomatic indicators determined to be affecting the user. In some instances, the time frame may be determined by the user. When additional responses to screening questions and/or additional data/information from the interface (e.g., one or more devices, an accelerometer, a pedometer, a geographical position sensing (GPC) module, an oximeter, a tactile sensor, etc.) is not received within the time frame, the user device may send a notification (e.g., a signal, a message, an email, a text, etc.) to the user device and/or the user that causes the user to provide additional responses to screening questions and/or additional data/information from the interface.

At 550, a user profile may be generated. The user profile may be generated by the user device. The user profile may include the score for each symptomatic indicator of the plurality of symptomatic indicators and the one or more signals. The user profile may associate the one or more scores with the one or more signals. The user profile may include at least the score for each symptomatic indicator of the plurality of symptomatic indicators. The user profile may include additional information related to the user, such as an electronic medical record, or military service record associated with the user.

The user profile may include any indication of a possible diagnosis of a health/medical issue associated with a symptomatic indicator of the plurality of symptomatic indicators. For example, the score for each symptomatic indicator of the plurality of symptomatic indicators and the one or more signals may be used to create a dataset. The dataset may be compared to datasets and/or information stored by a machine learning module. Comparing the dataset to datasets and/or information stored by the machine learning module may provide an indication of a possible diagnosis of a health/medical issue associated with a symptomatic indicator of the plurality of symptomatic indicators.

In some instances, scores derived from responses to the screening questions from multiple users and/or user devices may be used to create a training dataset. The training dataset may be used to train the machine learning module. When new data/information (e.g., clinical information, responses to screening questions, training datasets, etc.) is provided to the machine learning module, the network may update any stored data/information and adapt to any changing parameters (e.g., changes to a dataset, etc.) associated with the clinical data/information. The machine learning module may verify conclusions, diagnoses, and/or the like for accuracy and/or significance. The machine learning module may store test cases, appropriate outcomes and the relative occurrence of misidentification of the proper outcome and/or diagnosis. The machine learning module may establish a threshold of acceptable misidentifications or misdiagnoses.

The user profile may be stored. In some instances, the user device may store the user profile. In some instances a computing device (e.g., the computing device 104, server, etc.) may store the user profile. The user device may send the user profile (and/or data/information associated with the user profile) to the computing device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The computing device may receive a device identifier of the user device and a user identifier of the user. The computing device may use the device identifier of the user device and/or the user identifier of the user to authenticate the user device and/or user prior to storing the user profile.

In some instances, the user profile may be used to determine an alert condition. An alert condition may be determined based on a score associated for a symptomatic indicator of the plurality of symptomatic indicators. An alert condition may be and/or include an indication that the user is at risk for an emergency medical condition associated with a symptomatic indicator of the plurality of symptomatic indicators. The one or more scores may be compiled with additional medical information associated with each symptomatic indicator of the plurality of symptomatic indicators, and specific diagnosis, and prescribed services and/or treatments may be determined based on an alert condition. In some instances, the user device may determine the alert condition. For example, an application associated with the user device may determine the alert condition. In some instances, the computing device (and/or a device/system in communication with the computing device, etc.) may determine the alert condition based on the responses received from the user device.

To determine an alert condition, each score of the one or more scores may be accessed and/or analyzed according to one or more rules associated with each symptomatic indicator of the plurality of symptomatic indicators. For example, a rule associated with a symptomatic indicator for pain intensity may dictate that user scores that satisfy a threshold value may indicate an alert condition. Such that a score/value of 4 or greater (on an associated scale ranging from values 1-10) are considered to be clinically significant pain, and therefore indicative of an alert condition. An alert condition may be determined based on any rule associated with each symptomatic indicator of the plurality of symptomatic indicators.

In some instances, an alert condition may be determined by a machine learning module (e.g., the machine learning and analysis module 138/the machine learning-based classifier 230). For example, to determine a likelihood that a user will have a diagnosis of a health/medical issue associated with a symptomatic indicator of the plurality of symptomatic indicators, based on a score for the symptomatic indicator. The computing device and/or the user device may be in communication with (and/or comprise) a machine learning module (e.g., the machine learning and analysis module 138/the machine learning-based classifier 230). The machine learning module may include contemporaneous determined and stored user health data (e.g., from screening questions, the user profile, etc.). The machine learning module may comprise a collection of clinical data/information, such as historical symptoms, diagnoses and outcomes, along with time development of health/medical issues and symptomatic indicators. The clinical data/information may be coded and/or encoded and input into the machine learning module to populate and/or train the network with clinical data/information that may be used to determine, derived, and/or predict clinical, medical, health, and diagnostic outcomes, such as one or more alerts conditions. When new data/information (e.g., clinical information, responses to screening questions, etc.) is provided to the machine learning module, the network may update any stored data/information and adapt to any changing parameters associated with the clinical data/information. The machine learning module may verify conclusions, diagnoses, and/or the like for accuracy and/or significance. The machine learning module may store test cases, appropriate outcomes and the relative occurrence of misidentification of the proper outcome and/or diagnosis. The machine learning module may establish a threshold of acceptable misidentifications or misdiagnoses.

In some cases, when an alert condition (and/or any other condition associated with the plurality of screening questions) is determined, such as based on a score for a symptomatic indicator of the plurality of symptomatic indicators, a clinical device (e.g., the clinical device 107, a server, etc.) and/or an associated clinician may be determined. In some cases, the user device, based on determining the alert condition, may determine the clinical device and/or an associated clinician. For example, an application associated with and/or installed on the user device, based on determining the alert condition, may determine the clinical device and/or an associated clinician. In some cases, the computing device, based on determining the alert condition, may determine the clinical device and/or an associated clinician.

In some instances, the device identifier associated with the user device and/or the identifier associated with the user may be used to determine the clinical device and/or an associated clinician. For example, the device identifier associated with the user device and/or the identifier associated with the user may be used to determine the user profile. The user profile may indicate one or more clinical devices and/or clinicians associated with the user and/or user device. The one or more clinical devices and/or clinicians may each be associated with a symptomatic indicator (or one or more symptomatic indicators) of the plurality of symptomatic indicators. In some instances, the one or more clinical devices and/or clinicians may be determined based on a symptomatic indicator of the plurality of symptomatic indicators, such as based on a score derived from the plurality of medical questions for a particular symptomatic indicator of the plurality of symptomatic indicators. For example, each symptomatic indicator of the plurality of symptomatic indicators may be associated with one or more clinical devices and/or clinicians. The one or more one or more clinical devices and/or clinicians may be associated with a respective symptomatic indicator of the plurality of symptomatic indicators based on a clinical ability of a clinician associated with a clinical device to respond to, treat, diagnose, provide care for, and/or otherwise address any issue relating to and/or associated with the symptomatic indicator. The one or more clinical devices and/or clinicians may each be ranked based on a clinical ability of a clinician associated with a clinical device to respond to, treat, diagnose, provide care for, and/or otherwise address any issue relating to and/or associated with the symptomatic indicator. The rank associated with the clinical device and/or an associated clinician may be used to determine the clinical device and/or an associated clinician.

In some instances, the clinical device (e.g., the clinical device 107, a server, etc.) and/or an associated clinician may receive a notification (e.g., a signal, a message, an email, a text, etc.). In some cases, the user device, based on determining the alert condition, may send the notification to the clinical device. For example, an application associated with and/or installed on the user device, based on determining the alert condition, may send the notification to the clinical device. In some cases, the computing device, based on determining the alert condition, may send the notification to the clinical device. The notification may be sent to the clinical device via a long-range communication technique (e.g., Internet, cellular, satellite, and the like), via a short-range communication technique (e.g., BLUETOOTH®, ZigBee, Z-wave, near-field communication, infrared, etc.), and/or via any communication technique. The notification may be sent to the clinical device via an application associated with and/or installed on the user device, associated with the computing device, and/or the like. For example, the notification may be sent to the clinical device via an application program interface (API) associated with the user device, the computing device, the clinical device, and/or the like.

The notification may cause the clinical device to perform an action. For example, the notification may cause the clinical device to schedule an appointment (e.g., via an appointment scheduling system, online/web-based system, etc.) to meet with the clinician and/or a clinical staff associated with the clinical device. The notification may cause the clinical device to send data/information associated with the user of the user device to the user device and/or computing device. The data/information associated with the user of the user device may include appointment reminders, health and/or medical advice/instructions, information relating to a symptomatic indicator of the plurality of symptomatic indicators, and/or the like. The data/information associated with the user may include any data/information.

At 550, the user profile may be compared and/or associated with a plurality of profiles. The user device (and/or the computing device) may analyze data/information from a plurality of user profiles to determine commonalities among users. The user device (and/or the computing device) may compare demographics, responses to screening questions, and any other related data/information to determine, for particular symptom (symptomatic indicator) and/or health related issue, commonalities among users. Commonalities among users may be used to determine why a particular symptom (symptomatic indicator) and/or health related issue is occurring.

At 560, a target area may be determined. The user device (and/or the computing device) may determine a target area. For example, the user device (and/or the computing device), via machine learning (e.g., the machine learning and analysis module 138/the machine learning-based classifier 230), may analyze data/information associated with the user profile along with a plurality of user profiles associated with a plurality of users to determine and/or identify one or more target areas (e.g., geographical areas) where individuals likely may be associated with one or more symptomatic indicators. The user device (and/or the computing device) may predict and/or determine target areas (e.g., geographical areas) where there may a statistically relevant occurrence of a particular symptom (symptomatic indicator) and/or health related issue. For example, a target area may have a high rate of suicide. Other target areas may have a statistically high occurrence of people with cancer, blindness, deafness, or any other occupational or regional induced symptom and/or health issue. Target areas may identify any occurrence of a health, medical, or mental issue, as well as reasons for such occurrences. For example, the user device (and/or the computing device) may determine a target area where individuals experience a high rate of suicide. By correlating multiple user profile from users that may have worked, been stationed in, or otherwise been exposed to the target area, the user device (and/or the computing device) may determine why the target area has such a high suicide rate for such users. For example, responses to screening questions provided from users that may have worked, been stationed in, or otherwise been exposed to the target area may indicate that many post-traumatic stress inducing events occurred in the target area and thus results in a high suicide rate for the target area. The user device (and/or the computing device) may cause display of the target area or multiple target areas.

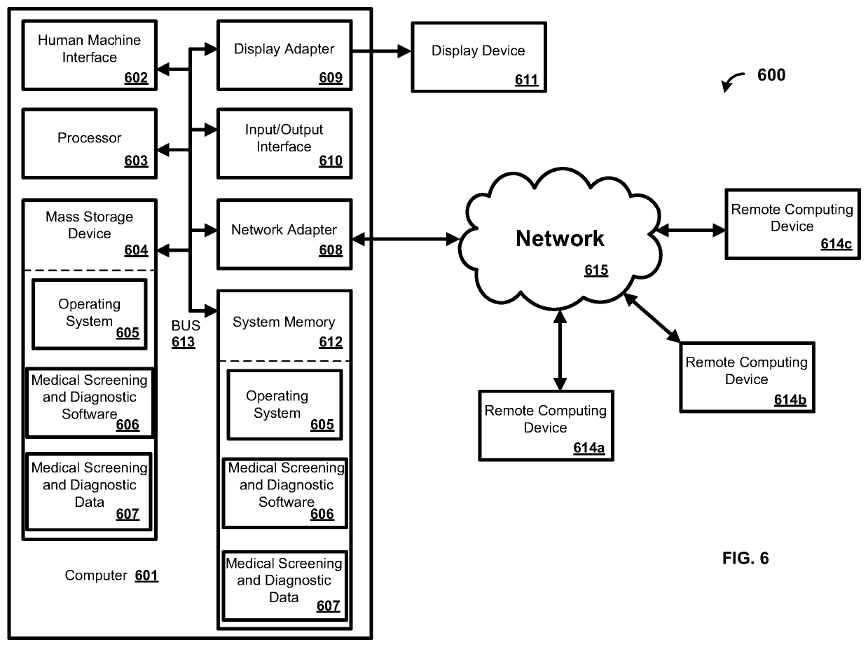

FIG. 6 shows an example computing device for implementing comprehensive symptom analysis. Any device described herein may be a computer 601. The computer 601 may comprise one or more processors 603, a system memory 612, and a bus 613 that couples various components of the computer 601 including the one or more processors 603 to the system memory 612. In the case of multiple processors 603, the computer 601 may utilize parallel computing.

The bus 613 may comprise one or more of several possible types of bus structures, such as a memory bus, memory controller, a peripheral bus, an accelerated graphics port, and a processor or local bus using any of a variety of bus architectures.

The computer 601 may operate on and/or comprise a variety of computer readable media (e.g., non-transitory). Computer readable media may be any available media that is accessible by the computer 601 and comprises, non-transitory, volatile and/or non-volatile media, removable and non-removable media. The system memory 612 has computer readable media in the form of volatile memory, such as random access memory (RAM), and/or non-volatile memory, such as read only memory (ROM). The system memory 612 may store data such as screening and diagnostic data 607 and/or program modules such as operating system 605 and screening and diagnostic software 606 that are accessible to and/or are operated on by the one or more processors 603.

The computer 601 may also comprise other removable/non-removable, volatile/non-volatile computer storage media. The mass storage device 604 may provide non-volatile storage of computer code, computer readable instructions, data structures, program modules, and other data for the computer 601. The mass storage device 604 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 604. An operating system 605 and screening and diagnostic software 606 may be stored on the mass storage device 604. One or more of the operating system 605 and screening and diagnostic software 606 (or some combination thereof) may comprise program modules and the screening and diagnostic software 606. Screening and diagnostic data 607 may also be stored on the mass storage device 604. Screening and diagnostic data 607 may be stored in any of one or more databases known in the art. The databases may be centralized or distributed across multiple locations within the network 615.

A user may enter commands and information into the computer 601 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 603 via a human machine interface 602 that is coupled to the bus 613, but may be connected by other interface and bus structures, such as a parallel port, game port, an IEEE 1394 Port (also known as a Firewire port), a serial port, network adapter 608, and/or a universal serial bus (USB).

A display device 611 may also be connected to the bus 613 via an interface, such as a display adapter 609. It is contemplated that the computer 601 may have more than one display adapter 609 and the computer 601 may have more than one display device 611. A display device 611 may be a monitor, an LCD (Liquid Crystal Display), light emitting diode (LED) display, television, smart lens, smart glass, and/or a projector. In addition to the display device 611, other output peripheral devices may comprise components such as speakers (not shown) and a printer (not shown) which may be connected to the computer 601 via Input/Output Interface 610. Any step and/or result of the methods may be output (or caused to be output) in any form to an output device. Such output may be any form of visual representation, including, but not limited to, textual, graphical, animation, audio, tactile, and the like. The display 611 and computer 601 may be part of one device, or separate devices.

The computer 601 may operate in a networked environment using logical connections to one or more remote computing devices 614a,b,c. A remote computing device 614a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 601 and a remote computing device 614a,b,c may be made via a network 615, such as a local area network (LAN) and/or a general wide area network (WAN). Such network connections may be through a network adapter 608. A network adapter 608 may be implemented in both wired and wireless environments. Such networking environments are conventional and commonplace in dwellings, offices, enterprise-wide computer networks, intranets, and the Internet.

Application programs and other executable program components such as the operating system 605 are shown herein as discrete blocks, although it is recognized that such programs and components may reside at various times in different storage components of the computing device 601, and are executed by the one or more processors 603 of the computer 601. An implementation of screening and diagnostic software 606 may be stored on or sent across some form of computer readable media. Any of the disclosed methods may be performed by processor-executable instructions embodied on computer readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
receiving, at a user device, a plurality of responses to a plurality of screening questions, wherein each screening question is associated with a symptomatic indicator of a plurality of symptomatic indicators;
determining, for each symptomatic indicator of the plurality of symptomatic indicators, based on the response to each of the plurality of screening questions, a score;
receiving, from an interface associated with the user device, one or more signals;
determining, based on the score for each symptomatic indicator of the plurality of symptomatic indicators and the one or more signals, a profile; and
storing the profile.

2. The method of claim 1, wherein storing the profile comprises:
establishing a communication session between the user device and an computing device system;
receiving, by the computing device, a device identifier of the user device and a user identifier associated with a user;
authenticating, based on a device identifier, the user device;
authenticating, based on the user identifier, the user; and
storing, based on authenticating the user device and the user, the profile.

3. The method of claim 1, further comprising encrypting at least one of, the device identifier, the user identifier, or the score for each response of the plurality of responses.

4. The method of claim 1 further comprising determining, based on at least one score for a symptomatic indicator of the plurality of symptomatic indicators, an alert condition for the user, wherein the alert condition comprises an indication that the user is at risk for an issue associated with the symptomatic indicator.

5. The method of claim 4, wherein determining, based on at least one score for a symptomatic indicator of the plurality of symptomatic indicators, an alert condition comprises determining, based on the score satisfying a threshold, the alert condition.

6. The method of claim 5, further comprising:
determining a clinician associated with a type of the alert condition; and
sending a message associated with the alert condition to the clinician.

7. The method of claim 1, wherein the user device comprises one or more of, a mobile phone, a tablet computer, a laptop computer, or a desktop computer.

8. The method of claim 1, wherein the interface comprises one or more of an accelerometer, a pedometer, a geographical position sensing (GPC) module, an oximeter, or a tactile sensor.

9. The method of claim 1, further comprising sending, based on a score of the plurality of scores satisfying a threshold, a notification.

10. The method of claim 1, further comprising sending, based on a signal of the one or more signals satisfying a threshold, a notification.

11. The method of claim 1, wherein presenting, via the user device, the plurality of screening questions comprises presenting the plurality of screening questions via an application running on the user device.

12. The method of claim 1, wherein the plurality of screening questions are associated with two or more of occupational and regional exposure, military service history, somatic symptoms, physical injury, illness, pain, post-traumatic stress disorder (PTSD) symptoms, behavior, depression symptoms, and social interactions.

13. The method of claim 1, wherein determining, for each symptomatic indicator of the plurality of symptomatic indicators, based on the response to each of the plurality of screening questions, the score comprises:
determining, based on the symptomatic indicator, a scale; and
scaling, based on the scale, the response to each of the plurality of screening questions, wherein the scaled response represents the score.

14. The method of claim 1, further comprising:
determining, for each of a plurality of user, a dataset comprising a score for each symptomatic indicator of the plurality of symptomatic indicators and one of, an indication of a possible diagnosis of a health issue related to the symptomatic indicator or an indication of no likely diagnosis of a health issue related to the symptomatic indicator;
determining, based on the dataset, a training dataset; and
training, based on the training dataset, a machine learning module to determine a likelihood that another user will have a diagnosis of an issue related to the symptomatic indicator based on the score for the symptomatic indicator.

15. A method comprising:
receiving, at a user device, a plurality of responses to a plurality of screening questions;
determining, for each response of the plurality of responses, a score;
receiving, via an interface of the user device, one or more signals;
determining, based on the score for each response of the plurality of responses and the one or more signals, a profile;
comparing the profile to a plurality of profiles, wherein each profile of the plurality of profiles is associated with a respective user of a plurality of users; and
determining, based on comparing the profile to a plurality of profiles, a target area, wherein each target area is associated with a symptomatic indicator.

16. The method of claim 15 further comprising, causing display of the target area;

17. The method of claim 15, wherein the user device comprises one or more of, a mobile phone, a tablet computer, a laptop computer, or a desktop computer.

18. The method of claim 15, wherein the interface comprises one or more of an accelerometer, a pedometer, a geographical position sensing (GPC) module, an oximeter, or a tactile sensor.

19. A method comprising:
determining first user data associated with a plurality of medical conditions;
determining second user data associated with the plurality of medical conditions, wherein the second user data comprises a plurality of user profiles each labeled as being indicative or not indicative of at least one of the plurality of medical conditions;
determining, based on the first user data and the second user data, a plurality of features for a predictive model;
training, based on a first portion of the second user data, the predictive model according to the plurality of features;
testing, based on a second portion of the second user data, the predictive model; and
outputting, based on the testing, the predictive model.

20. The method of claim 19, wherein the plurality of features for the predictive model comprise one or more pieces of clinical data or user health data.
